(19) United States
(12) Patent Application Publication
(10) Pub. No.: US 2020/0008757 A1
(43) Pub. Date: Jan. 9, 2020

(54) EARS: Emergency Alert Response System Monitoring Device

(71) Applicant: Ryan Phillip Bauch, Kalispell, MT (US)

(72) Inventor: Ryan Phillip Bauch, Kalispell, MT (US)

(21) Appl. No.: 16/504,342

(22) Filed: Jul. 8, 2019

Related U.S. Application Data
(60) Provisional application No. 62/695,815, filed on Jul. 9, 2018.

(51) Int. Cl. A61B 5/00; H04Q 9/02; H04W 4/80; G08B 3/10; G08B 6/00; G08B 7/06; G08B 21/02; G08B 21/12; A61B 5/0205; A61B 5/11

(52) U.S. Cl. CPC A61B 5/746; A61B 5/024; H04W 4/80; G08B 3/10; G08B 6/00; G08B 7/06; G08B 21/0277; G08B 21/12; A61B 5/02055; A61B 5/0022; A61B 5/7455; A61B 5/7405; A61B 5/1117; A61B 5/742; A61B 5/681; A61B 5/6804; A61B 2560/0242; A61B 2562/0219; A61B 2560/0214; H04Q 9/02

(57) ABSTRACT

A portable wearable health monitoring device which may be clipped to a user's clothing or strapped to their arm or area that doesn't affect a user's work or movement. The invention may monitor heart rate, tell time and date, monitor gas readings, and monitor temperature. It is equipped with a loud decibel alarm to alert other monitors linked with the device that there is an emergency. The alarm can be manually operated, automatically by heart rate reading being dramatically too low or too high, or by a connected person nearby to get help and notify others of emergency.

### CROSS-REFERENCE TO RELATED APPLICATION

The present application is related to and claims priority from prior provisional application Ser. No. 62/695,815, filed Jul. 9, 2018 which application is incorporated herein by reference.

### BACKGROUND OF THE INVENTION

### 1. Field of the Invention

The present invention relates generally to the field of health monitoring devices and more specifically relates to a portable wearable health monitoring device which may be clipped to a user's clothing or strapped to their arm or area that doesn't affect a user's work or movement. The invention may monitor heart rate, tell time and date, monitor gas readings, and/or monitor temperature. It is equipped with a loud decibel alarm to alert other monitors linked with the device that there is an emergency. The alarm can be manually or automatically operated to get help and notify others of emergency.

### 2. Description of the Related Art

When laboring in a confined or remote place such as a mine, oil field, or construction site, workers can quickly find themselves in a dangerous situation with no safety attendant within earshot and no way to easily call for assistance. In this kind of emergency, having a means to signal for help can mean the difference between life and death. A device capable of monitoring factors such as heart rate, ambient temperature, body temperature, detection of a fall and/or impact, and unsafe gas levels, and relaying data on these factors for a number of people in different locations to a centralized location for centralized monitoring, as well as being able to receive alarms to alert wearers to nearby dangers, could save lives in dangerous worksites.

Various attempts have been made to solve problems found in health monitoring devices art. Among these are found in: U.S. Pat. No. 8,321,004 to Moon et al; U.S. Pat. No. 8,961,413 to Teller et al.; U.S. Pat. No. 6,198,394 to Jacobsen et al; U.S. Pat. No. 6,579,231 to Eric T. Phipps; and U.S. Pat. No. 7,565,132Mourad Ben Ayed. This prior art is representative of portable health monitoring devices.

None of the above inventions and patents, taken either singly or in combination, is seen to describe the invention as claimed. Thus, a need exists for a reliable EARS: Emergency Alert Response System monitoring device to avoid the above-mentioned problems.

### BRIEF SUMMARY OF THE INVENTION

In view of the foregoing disadvantages inherent in the known health monitoring devices art, the present invention provides a novel EARS: Emergency Alert Response System (EARS). The general purpose of the present invention, which will be described subsequently in greater detail, is to provide a portable wearable health monitoring device, such as a watch, that may be clipped to a user's clothing or strapped to their arm or area that doesn't affect a user's work or movement. The invention may monitor heart rate, tell time and date, monitor gas readings, and monitor temperature. It is equipped with a loud decibel alarm to alert other monitors linked with the device that there is an emergency. The alarm can be manually operated to indicate distress, automatically triggered by a heart rate, temperature, or gas level reading being dramatically too low or too high, or by a fall or impact of the wearer or a nearby connected person, to get help and notify others of emergency. The monitoring device is linked to other devices through a mesh network. The features of the invention that are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures that accompany the written portion of this specification illustrate embodiments and method(s) of use for the presently claimed invention, EARS, constructed and operative according to the teachings of the present invention.

FIG. 1 shows a perspective view illustrating an EARS Mesh Network according to an embodiment of the present invention.

FIG. 2 shows a block diagram illustrating an EARS wearable device.

FIG. 3 shows a perspective view illustrating an EARS wearable device and an accompanying wristband and charging cord.

FIG. 4 shows a perspective view illustrating an EARS wearable device with user input buttons and clips for attaching the wearable device to the wearer.

FIG. 5 shows a perspective view illustrating an embodiment of the custom firmware run on a wearable device and/or the programmable centralized server for registering an EARS wearable device to the mesh network, when the wearable device is not connected.

FIG. 6 shows a perspective view illustrating an embodiment of the custom firmware on a wearable device and/or the programmable centralized server for monitoring one or more EARS wearable devices that are linked to specific wearers.

FIG. 7 shows a perspective view illustrating an embodiment of the custom firmware run on a wearable device and/or the programmable centralized server for monitoring the data from the wearable device's sensors for a specific wearer over time.

FIG. 8 shows a perspective view illustrating an embodiment of the custom firmware run on a wearable device and/or the programmable centralized server showing an alarm after one of the sensors has reported data outside of the acceptable range of values.

FIG. 9 shows a perspective view illustrating an embodiment of the custom firmware run on a wearable device and/or the programmable centralized server for registering an EARS wearable device to the mesh network, when the wearable device is connected.

The various embodiments of the present invention will hereinafter be described in conjunction with the appended drawings.

### DETAILED DESCRIPTION

As discussed above, embodiments of the present invention relate to a health monitoring device and more particularly to an EARS: Emergency Alert Response System.

The Emergency Alert Response System (EARS) provides additional safety to individuals working under hazardous conditions. EARS includes wearable devices that have the ability to communicate wirelessly with a network designed to provide immediate notifications to appropriate personnel in order to minimize response time in the event of an emergency.

The EARS wearable devices are intended to be similar to a rugged watch that has the following features:
Integrated sensors that monitor heart rate, skin temperature, ambient temperature, and detects falls and/or impacts.
Has the ability to send distress signals wirelessly to the EARS network. This signal can be triggered automatically by events related to the integrated sensors, or triggered manually by the user.
Displays relevant information to the user. This includes alerting the user of distress signals emanating from other nearby devices.
Must have low enough power consumption that the battery life allows for practical use of the device.
Functionality may be extended by additional wireless accessories (e.g. a wireless gas sensor).

The EARS network is a distributed mesh network including a central server with the following features:
Maintains constant contact with all devices in use on-site. The network should be reliable and have enough coverage to eliminate dead zones since any loss of contact will initiate an emergency alert.
The network should be able to determine the physical location of each device to a useful degree of accuracy (i.e. be able to provide the location of a distressed worker by room/zone/area).
Has the ability to send immediate alerts to appropriate personnel by means of EARS device alerts and also by means of external communications such as e-mails and cellular text messages.
May be integrated into currently existing on-site alarm and communications systems.
The network should be installed, configured, and tested on-site by a trained technician with minimal intrusiveness to the site.

The wireless communication network can be configured in several different ways and there are several existing wireless technologies that could be used in this application such as radio waves, Wi-Fi, Bluetooth, Zigbee, Z-wave, 6LowPAN, and LoRa. The wireless technology can be selected based on a number of factors, including power consumption, transmission range, operating frequency, cost, and complexity of implementation. The wireless communication network should ideally be a readily available product with very low power consumption, reasonable transmission ranges, relatively low implementation costs, and the ability to form a mesh network, such as Bluetooth Low-Energy (Bluetooth LE) or Zigbee.

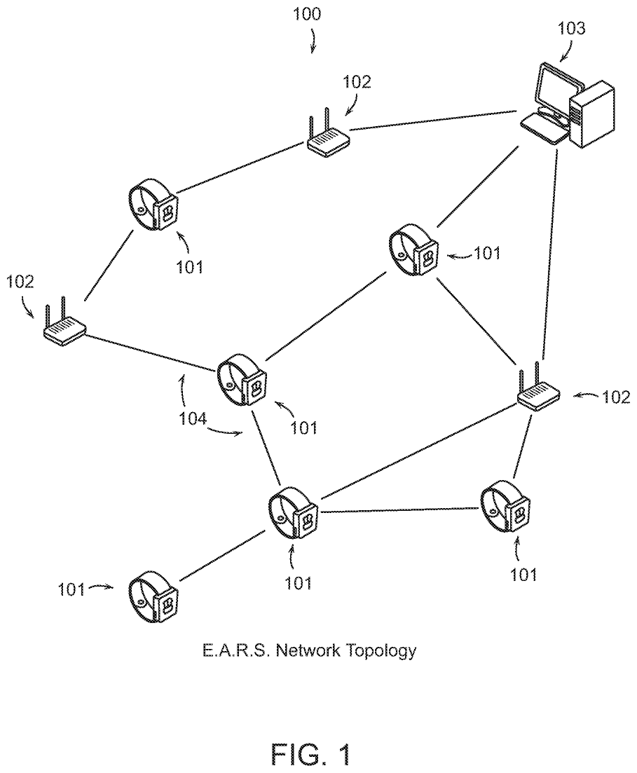

As shown in FIG. 1, the topology of the wireless mesh network 100 should allow wireless communication 104 between wearable devices 101, fixed wireless hubs 102 that are hard-wired for power, and a central server 103. The advantage of this topology is that it extends the range of reliable data transmission 104 while maintaining low power consumption. Since individual devices 101 do not need to use high power transmission to communicate directly with the server, they can transmit to the closest node 102 to them and the message will be relayed through the mesh 100 to the server 103. Also, by knowing where the devices 101 are in relation to each other and the closest node 102, the wearer's location can be approximated to reduce response time in an emergency.

EARS can also be integrated into existing on-site safety systems. Depending on the configuration of the existing system, alarms triggered by an EARS wearable device can be relayed to the on-site alarm system. Alarms triggered by the existing system can likewise be relayed to the EARS wearable devices. The wireless communication method can be customized to work with the existing on-site alarm system.

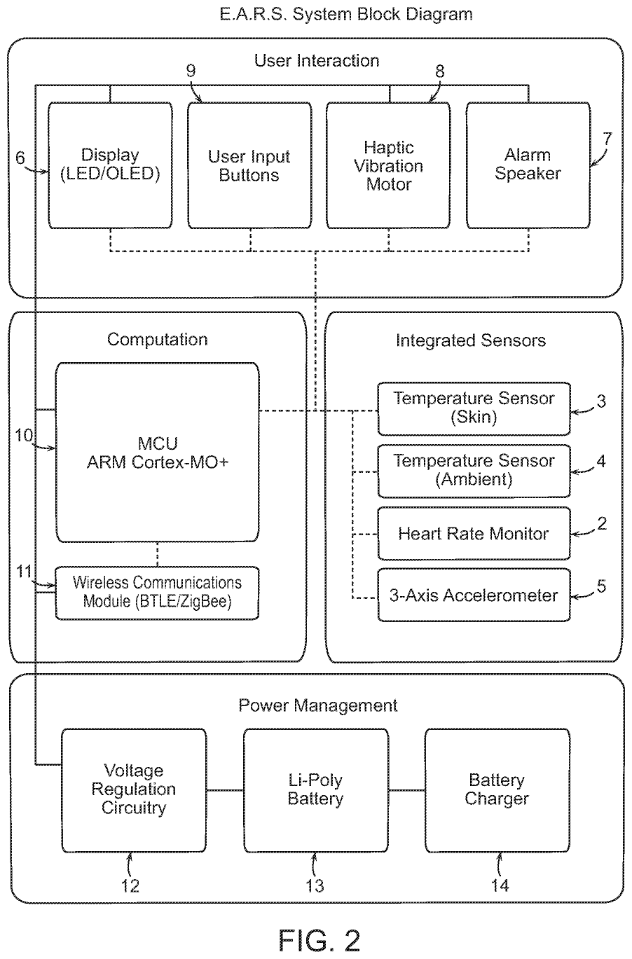

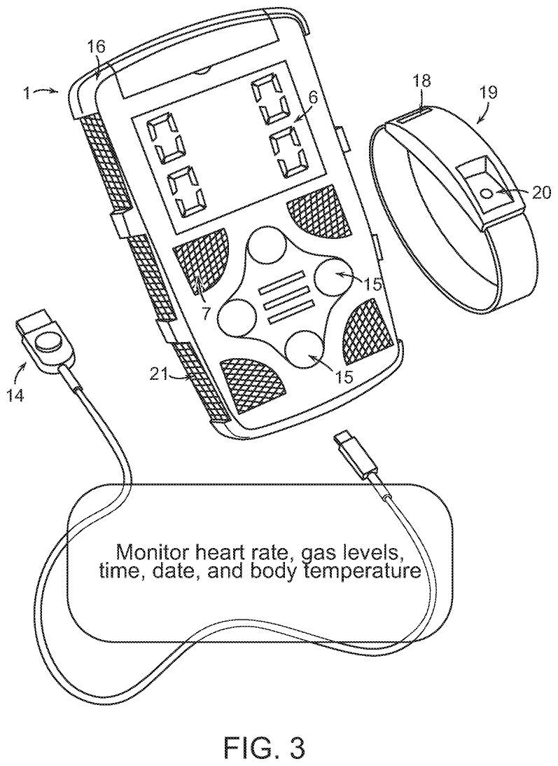

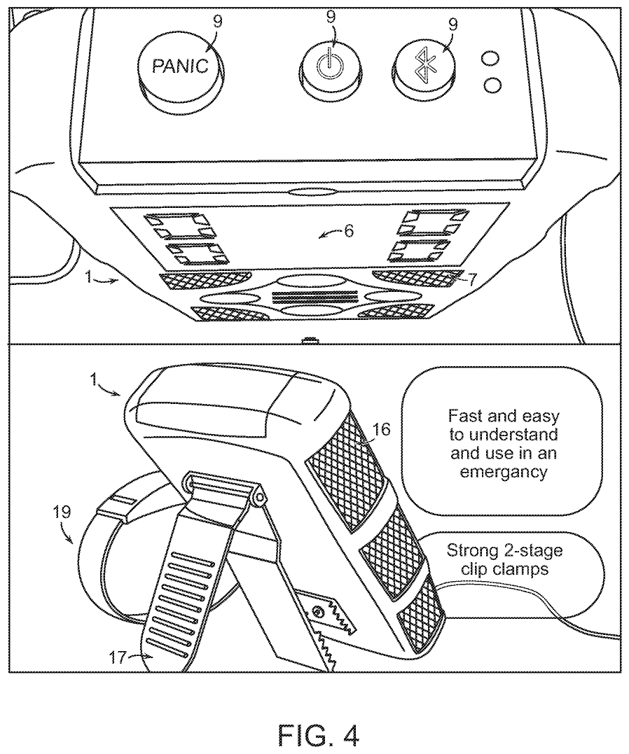

As shown in the block diagram in FIG. 2, and in FIGS. 3-4, the wearable device 1 needs to have an integrated heart rate sensor 2, an integrated skin/body temperature sensor 3, an ambient temperature sensor 4, and an integrated three-axis accelerometer 5 to detects falls and/or impacts. Each of these sensors is incorporated into the design of the wearable device 1 and has very low power consumption. The wearable device also has a simple Light-Emitting Diode (LED), Organic Light-Emitting Diode (OLED), or other type of screen 6 that displays information to the user. Information may also be communicated via speakers 7, haptic vibration motors 8, user input buttons 9, and/or status indicator lights 15. The wearable device also includes a microprosessor unit 10 and a wireless communications module 11 that allows data to be transferred to and from a server 103 through a wireless mesh network as shown in FIG. 1. The wearable device 101 also has voltage regulation circuitry 12 to regulate the battery 13 and the battery charger 14.

As shown in FIGS. 1-2, the integrated heart rate monitor 2 monitors the heart rate of the wearer and relays it through the mesh network 100 to the centralized server 103. If the wearer's heart rate exceeds or drops below a safe range, an alarm 7, 8 is triggered. Heart rate monitors have become an increasingly popular standard addition to smart watches and other wearable health devices, and there are a number of suitable readily available options, such as the BH1790GLC Optical Sensor for Heart Rate Monitor IC made by ROHM SEMICONDUCTOR.

As shown in FIGS. 1-2, the integrated temperature sensors 3, 4 monitor both the ambient temperature around the wearer, and the wearer's skin temperature and/or body temperature, and relay this information through the mesh network 100 to the centralized server 103. If either value falls outside an acceptable range for the wearer's safety, an alarm 7, 8 is triggered. Temperature sensors are commonly used in embedded systems in many applications, and a number of suitable, readily available options exist. One such option is the LM94021 CMOS integrated-circuit temperature sensor made by National Semiconductor.

As shown in FIGS. 1-2, incorporating a three-axis (or 3-axis) accelerometer 5 into the wearable device 101 enables the device 101 to monitor the wearer for falls and/or impacts and relay this information through the mesh network 100 to the centralized server 103. If a fall and/or impact is detected, an alarm is triggered. A number of suitable, readily available options already exist, such as the ADXL345 Digital Accelerometer made by Analog Devices.

As shown in FIGS. 3-4, the essential hardware components of the wearable device are similar to those of the current readily available smart watches and similar wearable devices that have become ubiquitous in recent years. The wearable device 101 has a body 1 to house the various sensors 2, 3, 4, 5, processor(s) 10, and other hardware components 6, 13, and attachment units 17, such as clips, clamp, and/or wrist straps, to tether the wearable device to the wearer. A number of other components may be included depending on the exact desired functionality, including user input buttons 9, status lights 15, haptic vibration motors 8, alarm speakers 7, voltage regulators 12, and other various electronic components 11. The body 1 may include a ribbed rubber housing 16 to provide a touch, secure grip, and provide water resistance, shock-resistance, dust-resistance, and durability. The body 1 may also have reflective stickers 21 placed on it to make it easier to locate in low-light situations. A wide variety of these are well known in the art and readily available on the market.

As shown in FIGS. 2-4, the wearable device 101 includes a display unit 6 that can be chosen from a wide selection of currently available display technologies to meet the specifically desired size, resolution, color palette, brightness, viewing angle, etc. One such readily available option is the 1.45″ AMOLED display made by US Micro Products. Ideally, the display unit will have a high-contrast screen and large user input buttons 9 for ease of use in low-light conditions. The alarm speakers 7 and vibrations produced by the motor(s) 8 will preferably produce different tones and/or vibration patterns for gas, heart rate, and emergency beacon alarms. The status lights 15 are preferably flashing, color-changing LED lights for easier locating of the wearable device 101.

As shown in FIG. 2, the wearable device 101 includes a programmable microprocessor (MCU) 10 that runs custom firmware and has full control over all of the other hardware components 2, 3, 4, 5, 6, 7, 8, 9, 11, 12, 13, in the wearable device. There are numerous readily available options for microprocessors, with new ones being continually developed. An example of a currently very widely used MCU that would be suitable is the ARM Cortex M0+.

As shown in FIGS. 2-4, the wearable device 101 includes a low-power embedded wireless communication system 11 to relay the data from the sensors to the centralized server 103 and receive any alarms 207 or other data in return. A number of suitable low-power, embeddable wireless communication systems are currently available, such as those discussed above. The built-in wireless communications module allows for the addition of other accessories that communicate using the same wireless mesh network. For example, a gas sensor module 18 can be clipped to a user's shirt, worn on a wristband, or otherwise attached to the wearer, and send data to the wearable device regarding the presence and levels of any gasses that may be hazardous to the wearer, including H₂S, LEL, O₂, and CO₂.

The wearable device 101 includes a compact rechargeable battery 13 that fits into the watch form body 1, and has a battery life long enough that the device is practical to use. Since the device will be worn throughout a typical workday and charged between shifts, a minimum of 12-hour battery life is required. A description of the typical power consumption of individual components that are required for the wearable device are shown below in Table 1, ¶24. One example of a currently available option is a 500 mAh rechargeable Li-Poly battery made by Shenzen Pkcell Battery Co., Ltd.

TABLE 1
Average Current Draw of Individual Components as Calculated from
Data Sheets of Suitable Commercially Available Components.

| | Average | |
|---|---|---|
| | Current | |
| | Draw (mA) | Notes |
| Microprocessor - ARM | 15 | Always on and Running |
| Cortex | | |
| Heart Rate Monitor | 0.2 | Always on |
| Temperature Sensors | 0.2 | Always on |
| OLED Display | 9 | On 50% of the time, |
| | | otherwise dim |
| Wireless Communications | 1.2 | Transmit data once per second |
| Module | | |
| Accelerometer | 0.05 | Always on |
| Voltage Regulators | 5 | |
| Total: | 30.65 | |

Certain components were not included in the power consumption estimate, such as the vibration motor or alarm speaker, as these components are unlikely to be used frequently, or possibly at all, during the course of a typical battery cycle.

As shown in FIG. 3, additional functionality can be provided by a breakaway wristband 19 that includes additional sensors 18 and connects with the wearable device by Bluetooth communications technology 20.

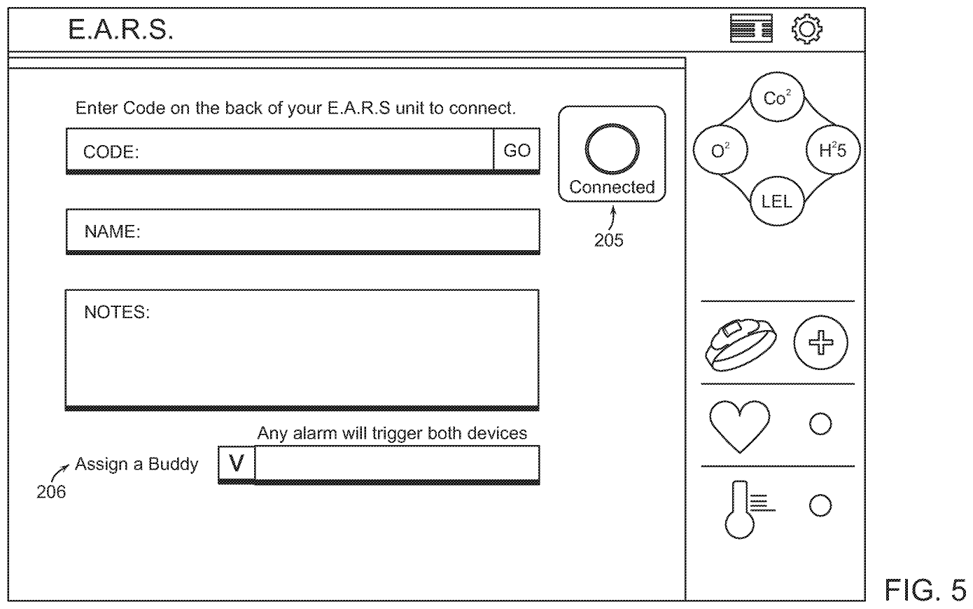

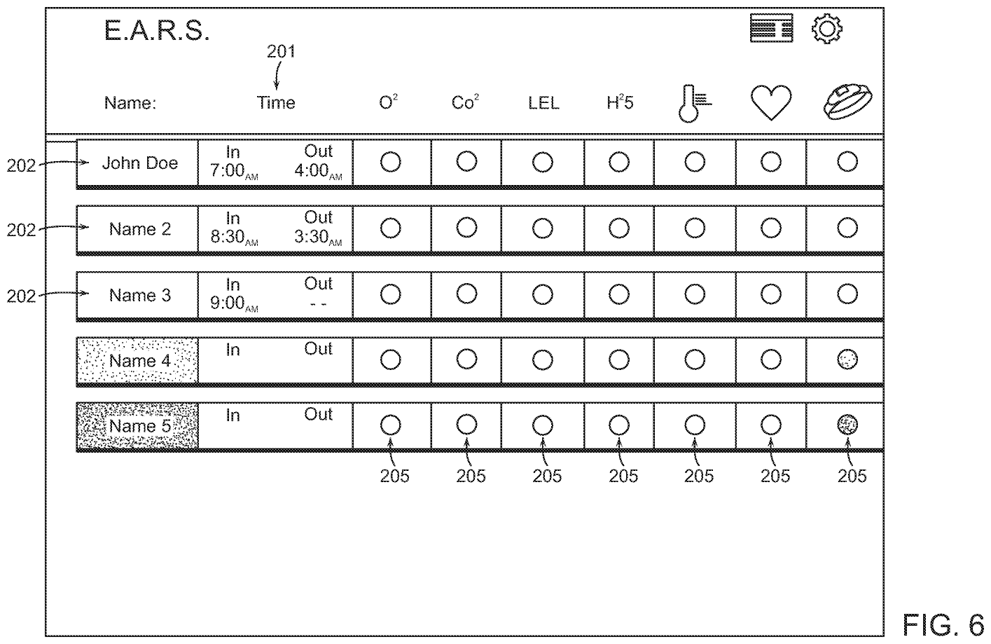

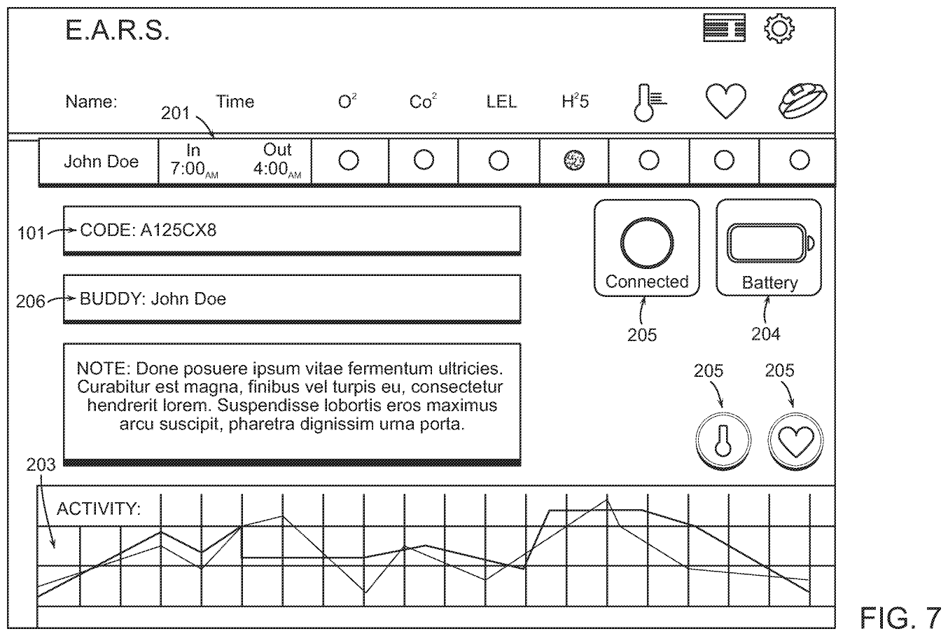

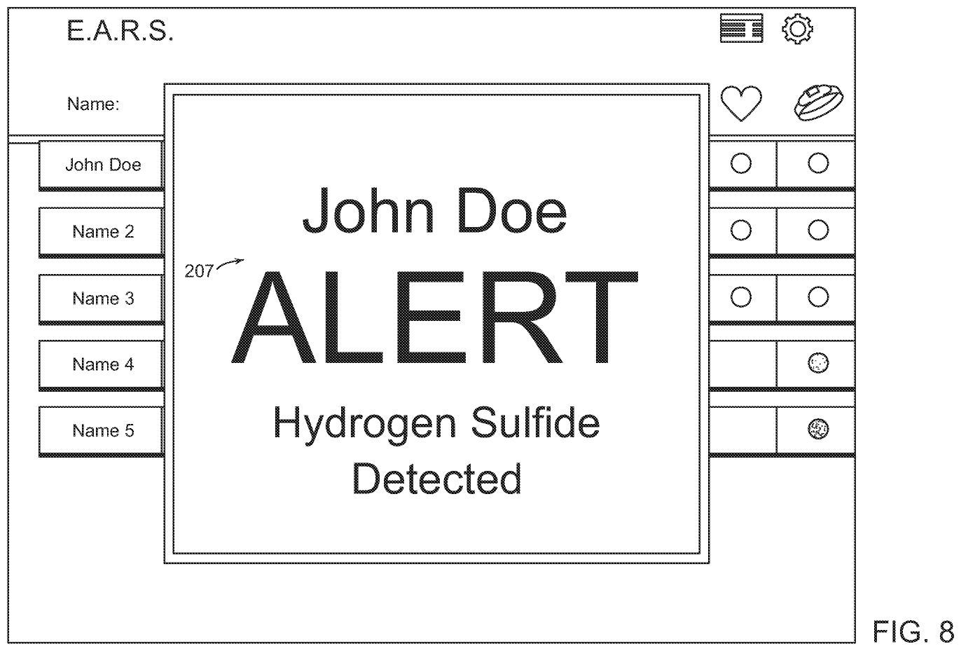

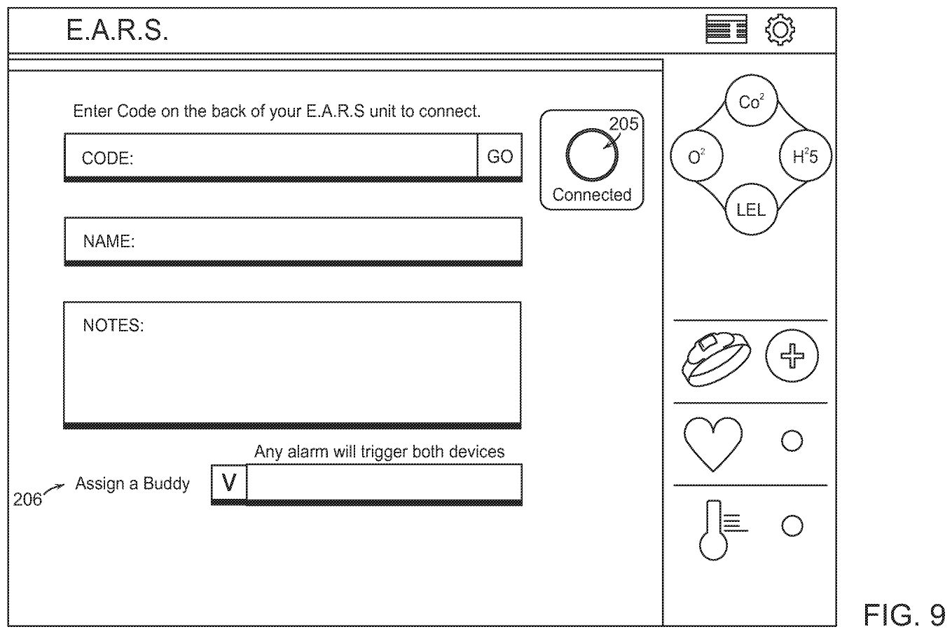

As shown in FIGS. 5-9, the wearable device 101 keeps track of the time and date 201 of each sensor reading for each wearer 202, so that, as shown in FIG. 7, the sensor data 203 can be graphed by the custom firmware. The device 101 also keeps track of the battery life 204, the connection to each of the sensors 205, and any alarms 207. Each device 101 can be linked to another wearable device 101 worn by a different wearer to establish a buddy system 206.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

The systems and techniques described herein can be realized or achieved in a number of ways, including digital electronic circuitry, application specific integrated circuits (ASICs), smartphone software, hardware, firmware, or any combination of the above. These various implementations of the claimed systems and techniques can be achieved through one or more computer programs, generally known as software, programs, software applications, and/or code.

The software can be implemented on a programmable system that includes one or more input devices, such as the wearable device, coupled to transmit and receive data from at least one programmable processor. The software includes machine instructions for the programmable processor and the input devices, and can be implemented in any appropriate programming language for the respective specifics of the programmable processor and input devices.

The systems and techniques claimed herein can be implemented using a computing system that includes a front end component, such as the wearable device that can interact with an implementation of the system and techniques described herein, and a back end component, such as a data server, or a middleware component, such as a fixed wireless hub, or any combination of a front end component with a middleware component and/or a back end component.

What is claimed is new and desired to be protected by Letters Patent is set forth in the appended claim:

1. A portable, wearable health monitoring device comprising:
(a) an integrated heart rate sensor;
(b) an integrated skin temperature sensor;
(c) an integrated ambient temperature sensor;
(d) a three-axis accelerometer;
(e) a wireless communications module including an antenna operably connected to send and receive signals wirelessly;
(f) a display unit;
(g) a body;
(h) a battery;
(i) one or more user input buttons;
(j) a vibration motor;
(k) a speaker operably connected to broadcast an alarm;
(l) voltage regulation circuitry;
(m) a microcontroller unit; and
(n) an attachment device.

2. The device according to claim 1, wherein the vibration motor is a haptic vibration motor.

3. The device according to claim 1, where in the three-axis accelerometer is operably connected to monitor falls, impacts, or falls and impacts.

4. The device according to claim 1, wherein the display unit is a Light-Emitting Diode display or an Organic Light-Emitting Diode display.

5. The device according to claim 1, wherein the battery is a rechargeable lithium polymer battery.

6. The device according to claim 1, wherein the attachment device is a pair of removable wrist straps or a double clamp.

7. The device according to claim 1, wherein the wireless communication module is a module selected from the group consisting of Bluetooth Low Energy, Wi-Fi, Zigbee, Z-wave, 6LowPAN, and LoRa.

8. The device according to claim 1, wherein the wireless communication module is a Bluetooth Low-Energy module.

9. The device according to claim 1, wherein the wireless communication module is a Zigbee module.

10. The device according to claim 1, wherein the wireless communication module further comprises:
(o) one or more status lights.

11. The device according to claim 1, the device further comprising:
(o) one or more gas sensors.

12. The device according to claim 11, wherein the gas sensors detect one or more gasses selected from the group consisting of H₂S, LEL, O₂, and CO₂.

13. The device according to claim 1, further comprising:
(p) a detachable module that connects wirelessly to the wireless communications module, wherein the detachable module includes one more additional sensors.

14. The device according to claim 1, wherein the speaker is operably connected to produce a different alarm tone for an alarm from each sensor.

15. The device according to claim 1, wherein the vibration motor is operably connected to produce a different alarm vibration for an alarm from each sensor.

16. The device according to claim 1, wherein the body of the device comprises a ribbed rubber housing.

17. The device according to claim 10, wherein the status lights are color-changeable LED lights that can be set to on, off, or flashing.

18. A portable, wearable health monitoring device comprising:
(a) an integrated heart rate sensor;
(b) an integrated skin temperature sensor;
(c) an integrated ambient temperature sensor;
(d) a three-axis accelerometer;
(e) a wireless communications module including an antenna operably connected to send and receive signals wirelessly, wherein the wireless communication module is a Bluetooth Low-Energy module or a Zigbee module;
(f) a display unit, wherein the display unit is a Light-Emitting Diode display or an Organic Light-Emitting Diode display;
(g) a body, wherein the body of the device comprises a ribbed rubber housing;
(h) a battery, wherein the battery is a rechargeable lithium polymer battery;
(i) one or more user input buttons;
(j) a vibration motor, wherein the vibration motor is a haptic vibration motor;
(k) a speaker operably connected to broadcast an alarm, wherein the speaker is operably connected to produce a different alarm tone for an alarm from each sensor;
(l) voltage regulation circuitry;
(m) a microcontroller unit;
(n) an attachment device;
(o) one or more status lights, wherein the status lights are color-changeable LED lights that can be set to on, off, or flashing;
(p) one or more gas sensors, wherein the gas sensors are selected from the group consisting of H₂S, LEL, O₂, and CO₂; and
(q) a detachable module that transmits data wirelessly to the wireless communications module via a Bluetooth Low-Energy module.

19. The device according to claim 18, wherein the detachable module includes one more additional sensors.

20. The device according to claim 18, wherein the speaker is operably connected to produce a different alarm tone for an alarm from each sensor.
